(19) United States
(12) Patent Application Publication
(10) Pub. No.: US 2019/0022315 A1
(43) Pub. Date: Jan. 24, 2019

(54) APPLICATOR

(71) Applicant: Simcro Limited, Hamilton (NZ)

(72) Inventors: Brendan Lance Udy, Te Kowhai (NZ); David Bain Calder, Ohaupo (NZ)

(73) Assignee: Simcro Limited, Hamilton (NZ)

(21) Appl. No.: 15/982,897

(22) Filed: May 17, 2018

Related U.S. Application Data
(63) Continuation of application No. PCT/NZ2016/050185, filed on Nov. 21, 2016.

(30) Foreign Application Priority Data
Nov. 19, 2015 (NZ) 714332

(51) Int. Cl. A61M 5/19; A61M 5/315; A61M 5/31

(52) U.S. Cl. CPC A61M 5/19; A61M 5/3159; A61M 5/31548; A61M 5/31578; A61M 5/3129

(57) ABSTRACT

An applicator has at least one barrel and a piston moveable within the at least one barrel between a variable first position and a second position. Movement of the piston towards the first position can draw a first fluid into the barrel, and movement of the piston towards the second position can force the first fluid out of the barrel. A fluid inlet valve allows the fluid to flow into the barrel at least under action of the piston, and a fluid outlet valve allows the fluid to flow out of the barrel. The piston is biased toward the first position and a dosage control part. An actuator is operable to move the piston to dispense the first fluid from the applicator.

### PRIORITY CLAIM

This application claims the benefit of New Zealand PCT application number PCT/NZ2016/050185, filed Nov. 21, 2016, which in turn claims the benefit of New Zealand application number NZ714332, filed Nov. 19, 2015, the contents of which are incorporated by reference.

### FIELD OF THE INVENTION

It is an object of a preferred embodiment of the invention to provide an applicator which will overcome or ameliorate at least one problem with such applicators at present, or which will at least provide a useful choice.

### BACKGROUND OF THE INVENTION

Animal remedies for sheep, cattle, swine and the like are applied by a number of methods including topical or “pour-on” application, oral application, injection and nasal infusion. Each of these is typically dispensed from a “pistol grip” style applicator.

Typically such applicators have a piston or plunger which can be reciprocated within a barrel by squeezing and releasing a first handle relative to a second handle. The liquid to be dispensed is drawn into the barrel through an inlet via a one way inlet valve when the plunger is withdrawn inside the barrel, and is dispensed through a nozzle (or a needle) via an outlet valve when the plunger is extended towards the outlet valve. Such an applicator is described in the applicant's New Zealand patent No. 521084.

In some cases the treatment given to the animal comprises two different liquids. The liquids may be mixed together within the applicator, or may be applied separately but at the same time. It may be desirable to control the dose of each liquid independently of the dose of the other.

Some applicators of the prior art comprise two parallel fluid flow systems which are operated by a single handle or trigger. In order to “prime” the applicator, the handle or trigger is operated repeatedly until each of the parallel systems is filled with a respective liquid. However, if one of the two systems fills sooner than the other (for example if the length of the conduits feeding the barrels are of different lengths, or if the parallel systems are set to express different doses), then wastage of at least one of the liquids can occur, as the priming action must be repeated until the last system to fill is ready for use. In many cases the liquids in question may be expensive, and so this waste may incur a significant expense for the user.

Many applicators of the prior art are provided with mechanisms for controlling the dose expressed by the applicator. NZ521084 describes a mechanism comprising a cylindrical dosage control part which is provided with a plurality of stopping ribs, each of a different length. Rotation of the dosage control part allows selection of which of the stopping ribs is engaged by a rib provided on the plunger, and therefor allows adjustment of the maximum stroke of the plunger.

Notably, the dosage control part encircles the plunger, and so the plunger must be of adequate length to allow the plunger to achieve its full stroke (dependent on the setting of the dosage control part) before the mechanism which actuates the plunger comes into contact with the dosage control part.

Often a user will need to treat a large number of animals. Accordingly, it is important that the applicator be as compact as possible so as to be easy to use and to avoid user fatigue.

Applicators may also require periodic servicing of various inlet and outlet valves in order to stay in good working condition. It is desirable for this servicing to be achievable as quickly and simply as possible. However, many applicators of the prior art have inlet and outlet valves located at a plurality of locations, requiring significant disassembly of the applicator for the valves to be serviced.

The reference to any prior art in the specification is not, and should not be taken as, an acknowledgement or any form of suggestion that the prior art forms part of the common general knowledge in any country.

### SUMMARY OF THE INVENTION

In a first aspect the present invention consists in an applicator comprising at least one barrel; a piston moveable within the at least one barrel between a variable first position and a second position, wherein, in use, movement of the piston towards the first position can draw a first fluid into the barrel, and movement of the piston towards the second position can force the first fluid out of the barrel; at least one fluid inlet valve to allow the fluid to flow into the at least one barrel at least under action of the piston, and at least one fluid outlet valve to allow the first fluid to flow out of the at least one barrel at least under action of the piston; biasing means to bias the piston toward the first position; a dosage control part to abut an abutment portion which in turn defines the first position of the piston and therefore the dosage of the first fluid; and piston actuating means operable to move the piston towards the second position to dispense the first fluid from the applicator, wherein a user may set the dosage of the applicator via the dosage control part, and deliver a dosage of the first fluid by the piston actuating means.

Preferably the abutment portion is on, or connected to, the piston.

Preferably the piston comprises a piston head and a piston rod connected to or integral with the piston head, wherein the piston rod comprises the abutment portion of the piston.

Preferably the dosage control part is slideable relative to the at least one barrel.

Preferably the dosage control part is slideable along an axis which is substantially parallel to a longitudinal axis of the barrel.

Preferably the dosage control part is externally accessible and slidable by a user.

Preferably the dosage control part is lockable in a selected position.

Preferably the piston comprises a lever portion which is accessible from an exterior of the applicator.

Preferably the lever portion allows a user to move the piston independently of the piston actuating means.

Preferably the first fluid enters the at least one barrel at or adjacent a first end of the at least one barrel, and the first fluid exits the at least one barrel at or adjacent the first end.

Preferably the at least one fluid inlet valve is adjacent one end of the at least one barrel, and the at least one fluid outlet valve is adjacent the same end of the at least one barrel.

Preferably the at least one fluid inlet valve and the at least one fluid outlet valve are adjacent the first end.

Preferably there is an outlet, wherein the outlet is at a second end of the at least one barrel, opposite to the first end.

Preferably the applicator comprises, a second barrel, a second piston moveable between a first position and a second position within the second barrel, wherein, in use, movement of the second piston towards the first position can draw a second fluid into the second barrel, and movement of the piston towards the second position can forces the second fluid out of the second barrel, at least one second fluid inlet to allow the second fluid to flow into the second barrel, and second biasing means to bias the second piston toward the first position, wherein the piston actuating means is operable to move both the first and second pistons towards the second position to dispense the first fluid and the second fluid from the applicator.

Preferably there is at least one second outlet valve to allow the second fluid to flow out of the second barrel.

Preferably the at least one fluid outlet valve may be in fluid communication with the second barrel also.

Preferably there is a second dosage control part to abut a second abutment portion which in turn defines the first position of the second piston.

Preferably the second piston comprises a second piston head and a second piston rod connected to or integral with the second piston head, wherein the second piston rod comprises the second abutment portion of the second piston.

Preferably the second dosage control part is slideable along an axis which is substantially parallel to a longitudinal axis of the first and second barrels.

Preferably there is a second lever portion which allows a user to move the second piston independently of the piston actuating means, and independently of the first piston.

Preferably the first fluid and the second fluid may be the same or different, and may be delivered in the same or different dosages.

In a second aspect the present invention consists in an applicator comprising a first barrel and a second barrel; the first barrel having a first piston and the second barrel have a second piston, the first piston being moveable between a variable first position and a second position within the first barrel, and the second piston being moveable between a variable first position and a second position within the second barrel, wherein, in use, movement of the pistons towards their respective first positions can draw respective fluids into each of the barrels, and movement of the pistons towards their respective second positions can force their respective fluids out of each of the barrels, for each barrel, at least one fluid inlet valve to allow the respective fluid to flow into the respective barrel when the respective piston moves towards the respective first position; at least one fluid outlet valve operable to allow the fluids to flow out of the barrels when the respective pistons move towards their respective second positions; biasing means to bias the first piston and second piston toward their respective first positions; a first dosage control part for the first barrel to abut a first abutment portion of the first piston to thereby define the first position of the first piston, a second dosage control part for the second barrel to abut a second abutment portion of the second piston to thereby define the first position of the second piston; piston actuating means operable to move the first piston and the second piston towards their respective second positions to dispense the respective fluids from the applicator, wherein a user may set the dosage of each of the first barrel and the second barrel via the respective first dosage control part and second dosage control part, and deliver a dosage of the first fluid and the second fluid by the piston actuating means.

Preferably there is a biasing means for each of the first piston and the second piston.

Preferably at least one fluid outlet valve is provided for each of the first barrel and the second barrel.

Preferably the at least one fluid outlet valve may operate as a common outlet valve for both the first barrel and the second barrel.

Preferably each dosage control part is slideable relative to the barrel.

Preferably the first dosage control part, and the second dosage control part are slideable along an axis which is substantially parallel to a longitudinal axis of the first barrel and the second barrel.

Preferably each of the first and second dosage control parts are lockable in their selected position.

Preferably the first piston comprises a first piston rod and a first piston head, wherein a first portion of the first piston rod is accessible from an exterior of the applicator, and second piston comprises a second piston rod and a second piston head, wherein a second portion of the second piston rod is also accessible from an exterior of the applicator.

Preferably each piston rod comprises a lever portion which allows a user to move the respective piston independently of the piston actuating means and independently of the other piston.

Preferably the first barrel and the second barrel each have a substantially ‘D’ shaped cross-section.

Preferably the first barrel and second barrel are back to back on the respective straight walls of their substantially “D” shaped cross section.

In a third aspect the present invention consists in an applicator comprising a barrel; a piston moveable between a first position and a second position within the barrel, wherein, in use, movement of the piston towards the first position draws a fluid into the barrel, and movement of the piston towards the second position forces fluid out of the barrel; at least one fluid inlet valve and at least one fluid outlet valve to allow fluid to flow into and out of the barrel; biasing means to bias the piston toward the first position; and piston actuating means operable to move the piston towards the second position to dispense a fluid from the applicator; wherein the at least one fluid inlet valve and at least one fluid outlet valve are located substantially adjacent each other.

Preferably the at least one fluid inlet valve and at least one fluid outlet valve are provided at a first end of the applicator, and the applicator has an outlet provided at an opposite second end of the applicator.

Preferably the applicator comprises a manifold portion in fluid communication with one end of the barrel, wherein the manifold portion is in fluid communication with the at least one fluid inlet valve and at least one fluid outlet valve.

In a fourth aspect the present invention consists in a method of using an applicator, comprising or including the steps of fluidly connecting supply of a first fluid to a manifold of the applicator, adjusting a dosage of a first piston with a first barrel of the applicator using a first dosage control part, where the first dosage control part is slidingly lockable along a major axis of the first barrel, the first dosage control part abutting a first abutment portion on the first piston which in turn varies a first position of the first piston and therefore the dosage of the first fluid, priming the first barrel using a first lever portion, forcing a first fluid from the first barrel by moving the first piston from the first position to a second position within the barrel, by use of a piston actuating means which, the first fluid moving from the first barrel via at least one fluid outlet valve, drawing the first fluid from the supply into the first barrel by the first piston moving from the second position back to the first position, the first fluid moving in via a first fluid inlet valve, the movement from the first position to the second position being biased, wherein a user may set the dosage of the applicator via the first dosage control part, and deliver a dosage of the first fluid by the piston actuating means.

In another aspect the present invention consists in an applicator substantially as herein described, with reference to any one of more the accompanying drawings.

In another aspect the present invention consists in an method of using an applicator substantially as herein described with reference to any one or more of the accompanying drawings.

Further aspects of the invention, which should be considered in all its novel aspects, will become apparent from the following description given by way of example of possible embodiments of the invention.

### BRIEF DESCRIPTION OF THE DRAWINGS

Preferred and alternative examples of the present invention are described in detail below with reference to the following drawings:

FIG. 1 is a perspective view of an applicator according to one embodiment of the present invention,

FIG. 2 is a side view of the applicator of FIG. 1,

FIG. 3 is a perspective view of the applicator of FIG. 1, with the housing and first handle removed,

FIG. 4 is a top view of the applicator of FIG. 1, with the housing and first handle removed,

FIG. 5 is a top view of the relative layout of the pistons and dosage control parts of the applicator of FIG. 1,

FIG. 6 is a perspective view of the second handle, pistons and dosage control parts of the applicator of FIG. 1, with the second dosage control part shown in partially exploded form,

FIG. 7 shows the relative layout of the second handle, conduits and valves of the applicator of FIG. 1,

FIG. 8 is a partially exploded perspective view of the first dosage control part,

FIG. 9 shows a side view of a further embodiment of the present invention, where the manifold is located at the top rear of the body,

FIG. 10 shows the rear view of the embodiment of FIG. 9,

FIG. 11 shows the plan view of the embodiment of FIG. 9,

FIG. 12 shows a rear isometric view of the embodiment in FIG. 9,

FIG. 13 shows an exploded view of the embodiment of FIG. 9,

FIG. 14 shows the rear end of the exploded view of FIG. 13,

FIG. 15 shows the middle portion of the exploded view of FIG. 13, and

FIG. 16 shows the front end of the exploded view of FIG. 13.

### DETAILED DESCRIPTION OF THE PREFERRED EMBODIMENT

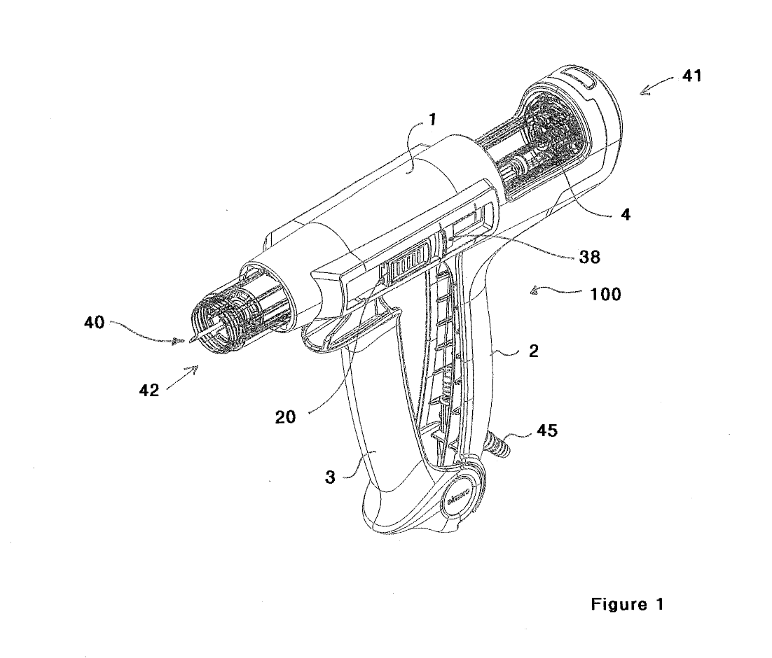

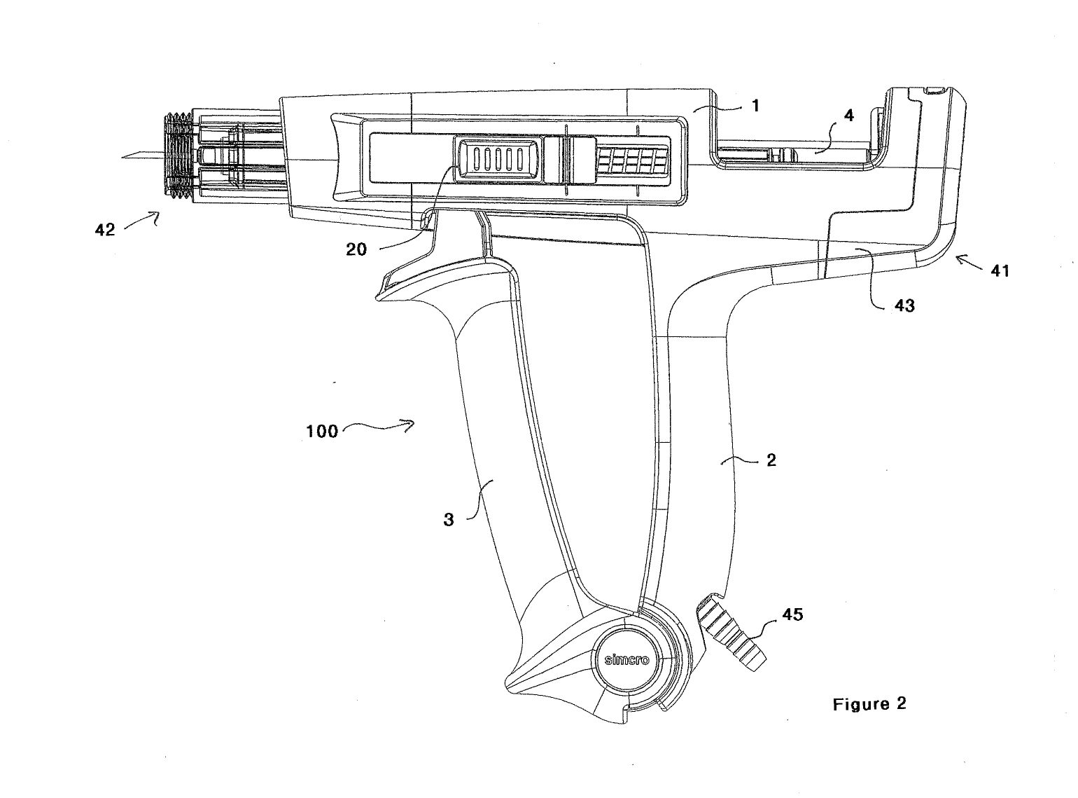

Referring first to FIGS. 1-4, an applicator according to one embodiment of the present invention is generally referenced by arrow 100.

The applicator described and in the embodiments has two barrels and associated components to deliver two fluids, or a greater volume of a single fluid from the two barrels. It is to be understood that a single barrel applicator, though not shown, is also an embodiment of the present invention. Such a single barrel applicator may be buildable from the dual barrel applicator shown, for example by disassembly and replacing the barrel, manifold and associated components with a single barrel. In this way it may be made with the same body, and second handle, outlet but have a single barrel and piston and adjusters so that where possible common parts may be used.

Alternatively all, or a majority of, the components may be specific to the single barrel applicator.

Also an applicator with more than 2 barrels is an embodiment of the present invention, though not shown. A person skilled in the art would understand how such an applicator could have three or more barrels arranged equally about the longitudinal centre line, each with its own piston, valving, adjustment and priming levers.

The applicator 100 comprises a housing 1 which is connected to or integral with a first handle 2, and has a first end 41 and second end 42. A second handle 3 is pivotally connected at a lower end to the first handle 2. In use, movement of the second handle 3 relative to the first handle 2, and thus the body or housing 1 activates the applicator 100, as is described further below, to deliver fluid(s) from an outlet 40 at the second end 42. The second handle 3 drives a piston, one per barrel 4, to draw fluid into the barrel, and expel it out again. The dosage of the barrel can be adjusted using dosage control parts 20, and in the case of two barrels second dosage control part 21. To prime the barrel, or barrels there is a lever portion 38, and 39 when two barrels, to fill the barrel(s) with fluid. This is so there is minimal wastage when there are two or more barrels.

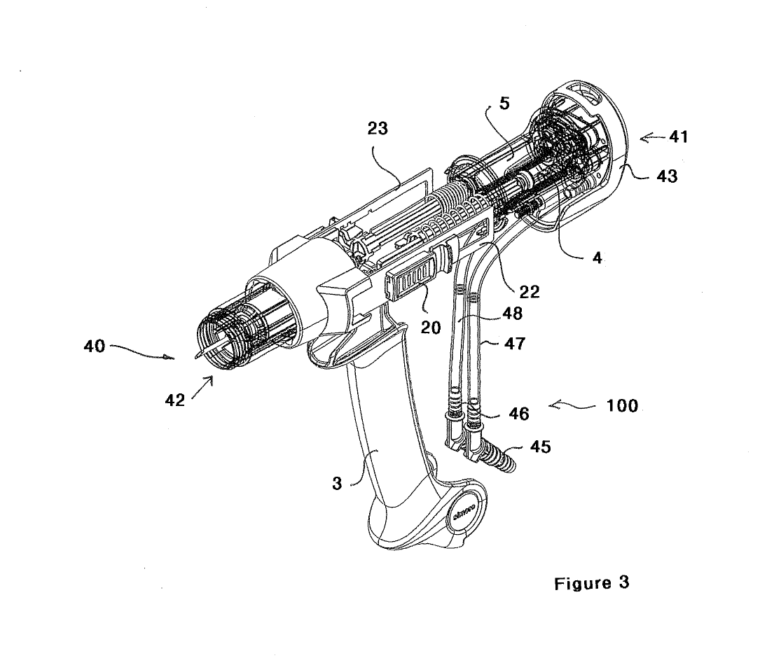

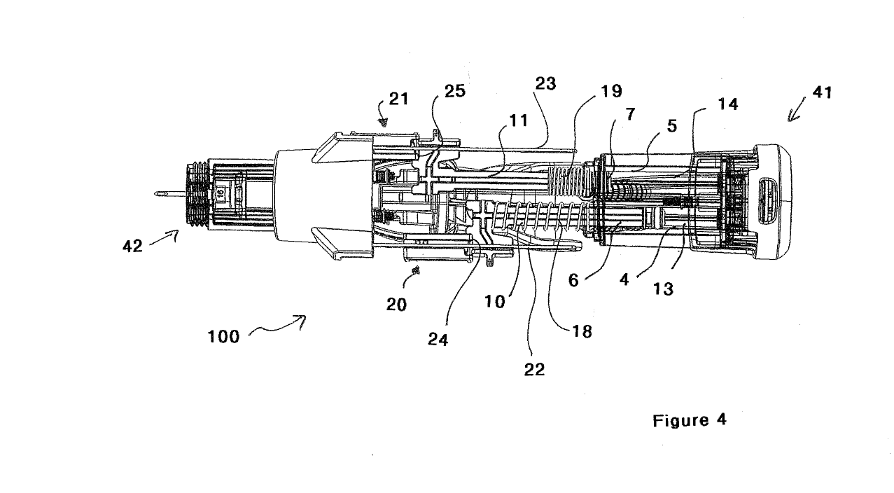

Referring next to FIGS. 3 and 4 in particular, the applicator 100 has two barrels 4, 5 which are mounted to or integral with the housing 1 (not shown here). The barrels 4, 5 are preferably adjacent to and parallel with each other. In the embodiment shown the barrels 4, 5 have the same diameter. However, in many embodiments the first barrel 4 will have a different diameter to the second barrel 5. The barrels 4, 5 shown in FIGS. 3 and 4 are substantially circular in cross-section. However, in some embodiments (not shown) the barrels may be substantially “D” shaped in cross-section, in order to reduce the overall width of the applicator.

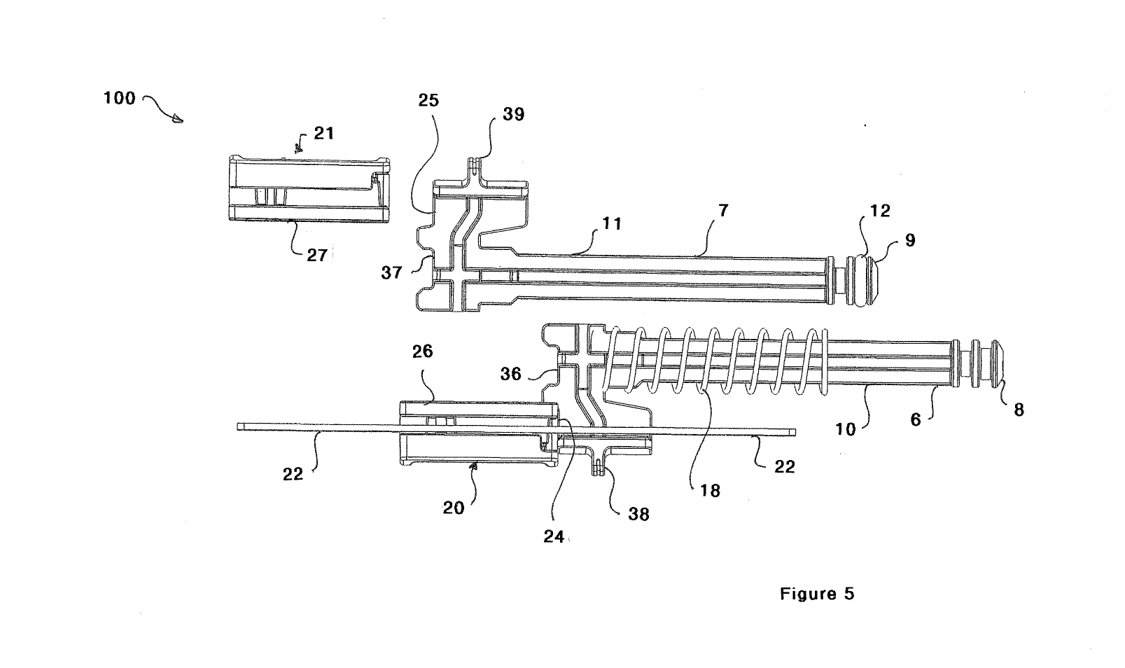

As shown in FIGS. 4 to 6, each barrel 4, 5 is provided with a respective piston 6, 7, the pistons 6, 7 each comprising a head 8, 9 which is connected to or integral with a respective piston rod 10, 11. The piston heads 8, 9 are provided with suitable seals 12 (the seal from the first piston 6 is omitted in FIG. 5) to seal against an inner wall of the respective barrel 4, 5 to thereby form a chamber 13, 14. As will be familiar to those skilled in the art, the volume of the chambers 13, 14 varies as the pistons 6, 7 are moved within the barrels 4, 5 between a variable first position and a second position, the volume being greater when the pistons 6, 7 are in the first position. This is shown for example in FIG. 4, where first piston 6 is adjusted (explained below) so that its first position, which is variable, is further into its travel when compared to the second piston 7 which is adjusted (explained below) so that its first position, which is also variable, is less further into its travel.

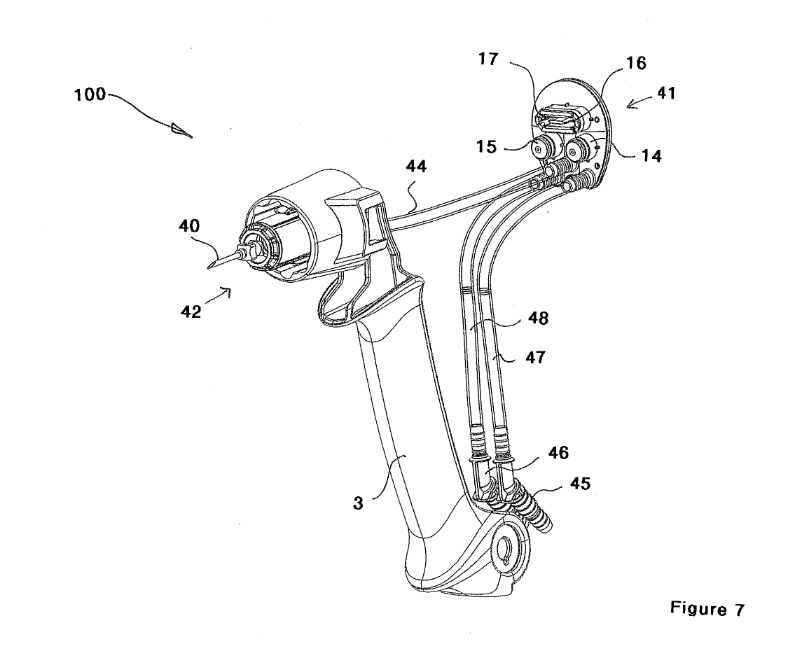

Referring next to FIGS. 4 and 7, suitable inlet valves 14, 15 and outlet valves 16, 17 are provided for each barrel 4, 5. The inlet and outlet valves are one-way valves. In the embodiment shown the inlet and outlet valves are provided at one end of the respective barrels 4, 5, but in other embodiments (not shown) the inlet and outlet valves may be provided in conduits which are in fluid communication with the barrels 4, 5. In another embodiment (not shown) the barrels 4, 5 may share a common outlet valve or a plurality of common outlet valves.

Movement of the pistons 6, 7 towards their respective first positions (to the left when seen from above in FIGS. 4 through 7) draws fluid into the respective barrels through the respective inlet valves 14, 15, and movement of the pistons 6,7 towards the second position (to the right when seen from above in FIGS. 4 through 7) forces fluid from the barrels through the outlet valves 16, 17.

Referring back to FIGS. 4 to 6, each piston 5, 6 is provided with a biasing means, typically a spring 18, 19 (only one spring shown in FIG. 5), to bias the piston 4, 5 towards the first position.

The travel of the piston 4, 5 under the influence of the biasing means 18, 19 (and therefor the maximum volume of each chamber) is limited by respective dosage control parts 20, 21. The dosage control parts 20, 21 thereby define the first position for the pistons. In the embodiment shown each dosage control part 20, 21 is an assembly and is provided as an adjustable stop which is slideably engaged with a respective slotted component 22, 23 which is attached to or integral with the body 1.

Referring next to FIGS. 4 to 6, each piston rod 10, 11 has an abutment portion 24, 25 which abuts the respective dosage control part 20, 21. Note that in FIG. 5, dosage control part 21 is shown spaced apart from abutment portion 25 for clarity.

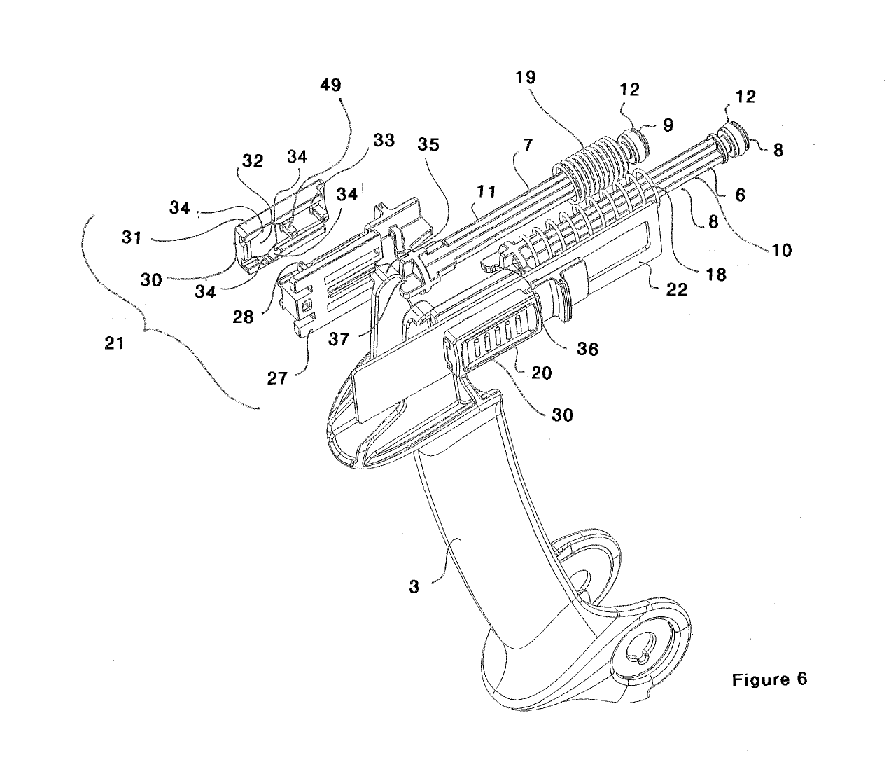

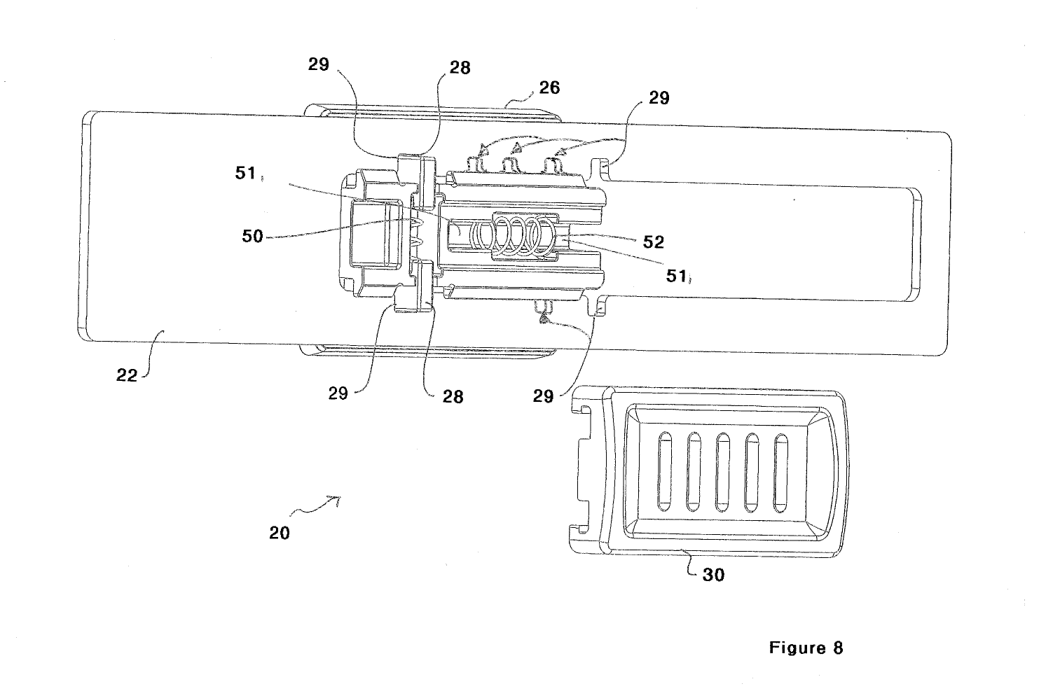

Referring next to FIGS. 6 and 8, in the embodiment shown the dosage control parts 20, 21 comprise in the embodiment shown an assembly of a dosage control part body 26, 27, each provided with at least one, and more preferably a pair of moveable locking tabs 28 which engage complementary notches 29 provided in the sides of the slot of the slotted component 22, 23 (best seen in FIG. 8). The tabs 28 are outwardly biased, using a tab spring 50. This tab spring (or springs if needed) can act on a suitable part of the dosage control part body, or for example where there are two locking tabs as shown in FIG. 8, may engage on each other to bias the tabs outwardly (vertically in the case of FIG. 8)

The locking tabs 28 can be moved inwardly (described below) to disengage the notches 29. The slotted component is shown in FIG. 8 as having eight notches 29. However, in other preferred forms as needed the notches 29 may be anywhere along the length of the slotted component 28. This will provide varying levels of adjustment and therefore differing first positions to provide varying dosages as needed. In other form, where there are two locking tabs 28 present, the notches 29 may be staggered on either side to provide additional resolution to the adjustable first position to in turn provide a greater variety of doses. In this embodiment each locking tab may alternatively engage a notch on one side, and then the other locking tab will engage another notch further along on the other side. Suitable markings may be provided on the dosage control part and the body, for example an indicator mark on the dosage control part, for example the button 31, and dosage volume markings (for example in millilitres) on the body, or vice versa.

The slotted components 22, 23 and locking tabs 28 are in the preferred embodiment made from a tough resilient material to withstand repeated adjustments and repeated stops and starts of the respective piston movements. In one form they may be made of metal, preferably no-ferrous, such as stainless steel.

Shown in FIG. 6 each dosage control part 20, 21 is provided with an exterior button 30, 31 which has a recess 32 on an inward face 33 which is provided with angled sides or ramps 34. The ramps as shown in FIG. 6, taper inwardly from the interior toward the button 30, 31 exterior. This is such that the angled sides 34 of the recess 32 move the tabs 28 inwardly and therefore out of engagement with the respective notches 29 when the button 30, 31 is moved sideways that is, inward, relative to the body. In an alternative embodiment (not shown) the slot may not be provided with notches. In this embodiment each dosage control part may be held in a required position by alternative means, such as friction.

The button is further biased outwardly from the body 1. Present on the interior side of the dosage control part, and in this embodiment shown the interior side of the button 30 and 31, are ramped extensions 51 (shown in FIG. 6). These taper inward from the interior side of the button, towards its exterior. Residing between them is a button spring 52 (seen in FIG. 8), which bears on the ramped extensions 51 to bias the button outwardly.

Referring next to FIGS. 1, 5, 6 and 7, in use, fluid is expressed from the applicator 100 by squeezing the first and second handles 2, 3 towards each other. This will move the second handle 3 along the body 1 to actuate the pistons 6 and 7. As is best seen in FIGS. 5 and 6, movement of the second handle 3 causes an engagement portion 35 of the handle (typically provided at an opposite end of the handle 3 to the pivotal connection with the first handle 2) to move into engagement with a second abutment portion 36, 37 of each piston 6, 7. Shown in FIG. 5 the abutment portions 36, 37 are at differing location due to the dosage they are adjusted to.

Depending on the relative settings of the dosage control parts 20, 21, the engagement of the engagement portion 35 with the second abutment portions 36, 37 therefore may not occur simultaneously. For example, in the embodiment shown in FIGS. 5 and 6 the engagement of the engagement portion 35 with second abutment portion 37 of piston 7 will occur first, and fluid will begin to be expressed from the barrel 5 associated with piston 7 before any fluid is expressed from the barrel 4 associated with piston 6. In some embodiments it may be possible to adjust the dosage control parts 26, 27 such that substantially no fluid is expressed from the respective barrel. In this way a dual barrel applicator may be used to express only one fluid, if required.

As can be seen from FIGS. 4 and 6, in contrast with the dosage control mechanisms of the prior art, the engagement portion 35 is able to move past the dosage control parts 20, 21 during actuation of the pistons 6, 7. This allows the length of the piston rods 10, 11 (and therefore the entire applicator) to be reduced, as otherwise the piston rods 10, 11 would need to be longer in order to allow each piston to move over its full stroke.

As best seen in FIG. 5, one or both of the piston rods 10, 11 are provided with a lever portion 38, 39 which extends though the slot and is accessible from the exterior of the applicator 100. This lever portion 38, 39 can be used to actuate the respective piston 6, 7 independently from the handle 3 or the other piston. This is useful where the applicator 100 must be primed before use. To prime the applicator 100 the handle 3 may be operated until one of the barrels 4, 5 is full. The barrels 4, 5 may be transparent or translucent to allow the user to see when this has occurred. When the first barrel to fill has filled, the operator may use the lever portion 38, 39 of the piston associated with the other barrel to actuate that piston until the second barrel is full. This allows the device to be primed with minimum waste.

Referring next to FIGS. 1, 3 and 7, in preferred embodiments the inlet valves 14, 15 and outlet valves 16, 17 for each barrel 4, 5 are provided at a first end 41 of the applicator, and an outlet 40 is provided at an opposite second end 42 of the applicator. In preferred embodiments each barrel 4, 5 has an expanded portion or a manifold portion at one end to allow the inlet and outlet valves to be provided at the same end of the barrel. Grouping of the inlet and outlet valves together in this way allows easy access to all of the valves by removing a single cover 43, rather than requiring more substantial disassembly of the applicator. In preferred embodiments the outlet valves 16, 17 feed a manifold (not shown) which combines the fluids from the barrels 4, 5 before a single outlet conduit 44 feeds the combined fluid to the outlet 40. If required the manifold and/or outlet conduit 44 may be provided with suitable vanes or other formations (not shown) to ensure that the fluids from the barrels 4, 5 are properly mixed together prior to being expressed through the outlet. In other embodiments (not shown) the outlet fluids may be prevented from mixing until reaching the front or first end 42 of the applicator 100 by providing separate outlet conduits (a duplication of conduit 42) from each barrel to the outlet 44—the dual conduits joining using a “Y” join (not shown) at or near the outlet. This embodiment reduces the mixed volume of the two products prior to application. Minimal mixed product may be the preferred practice, depending on the specific products to be combined.

The inlet valves 14, 15 are connected to respective inlet connections 45, 46 by inlet conduits 47, 48. In the embodiment shown the inlet connections are 45, 46 are provided at the base of the first handle 2. In another embodiment (not shown) the inlet connections 45, 46 may be provided at the second end 42 of the applicator.

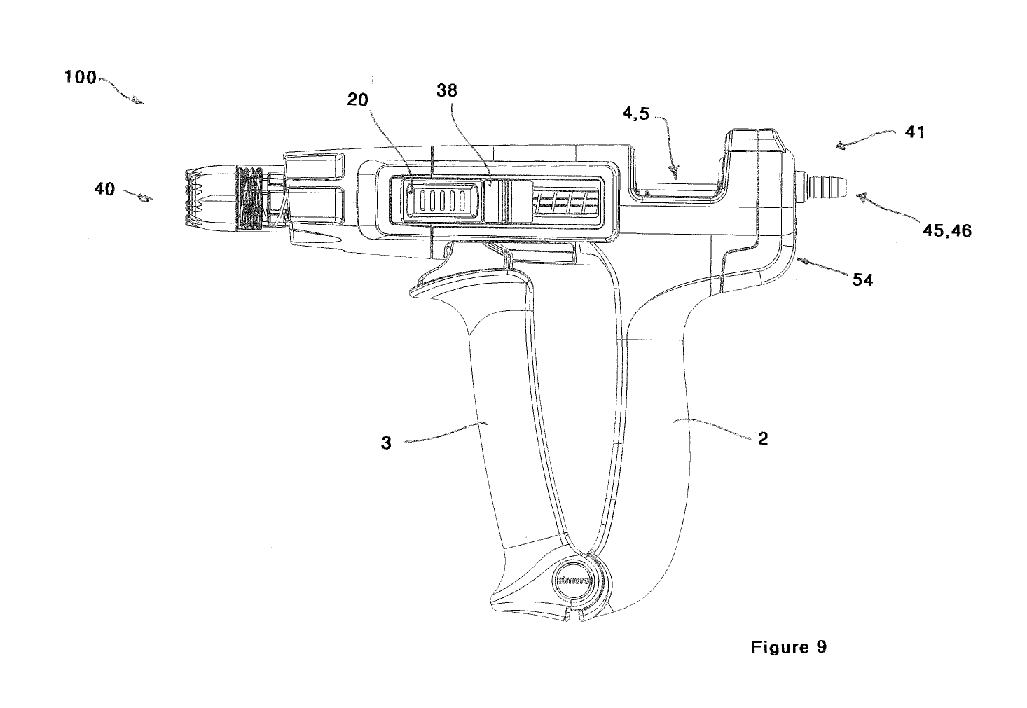

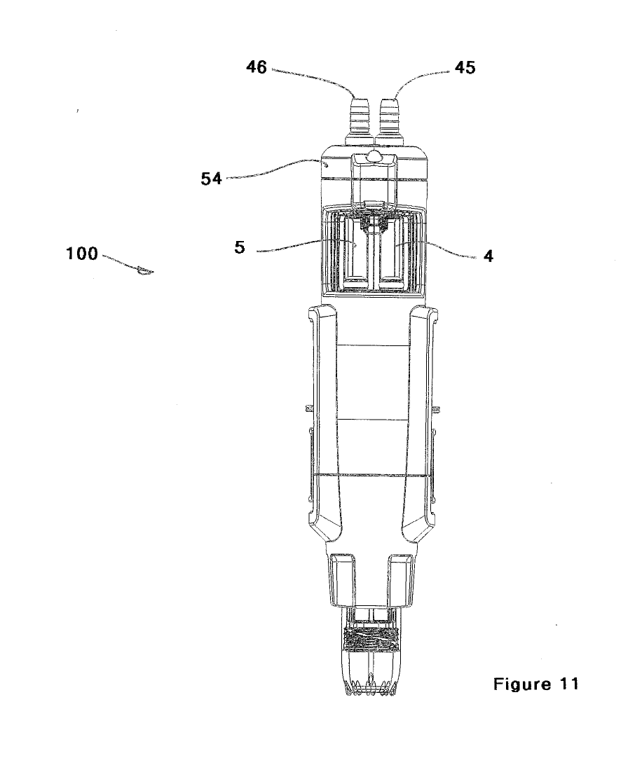

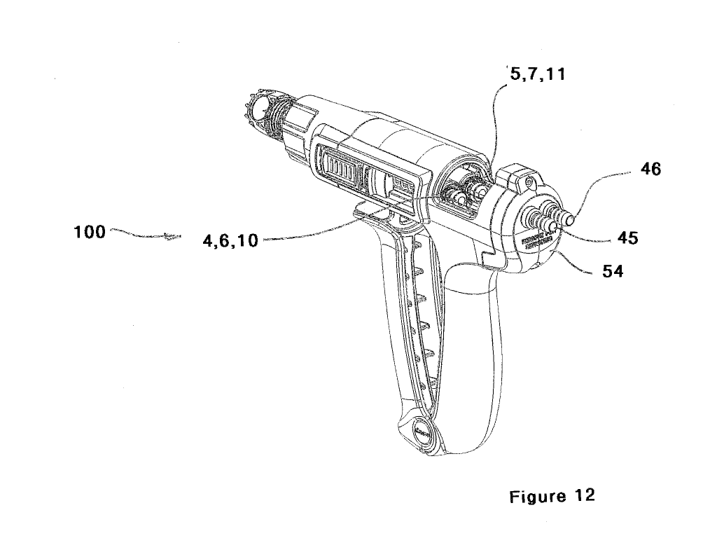

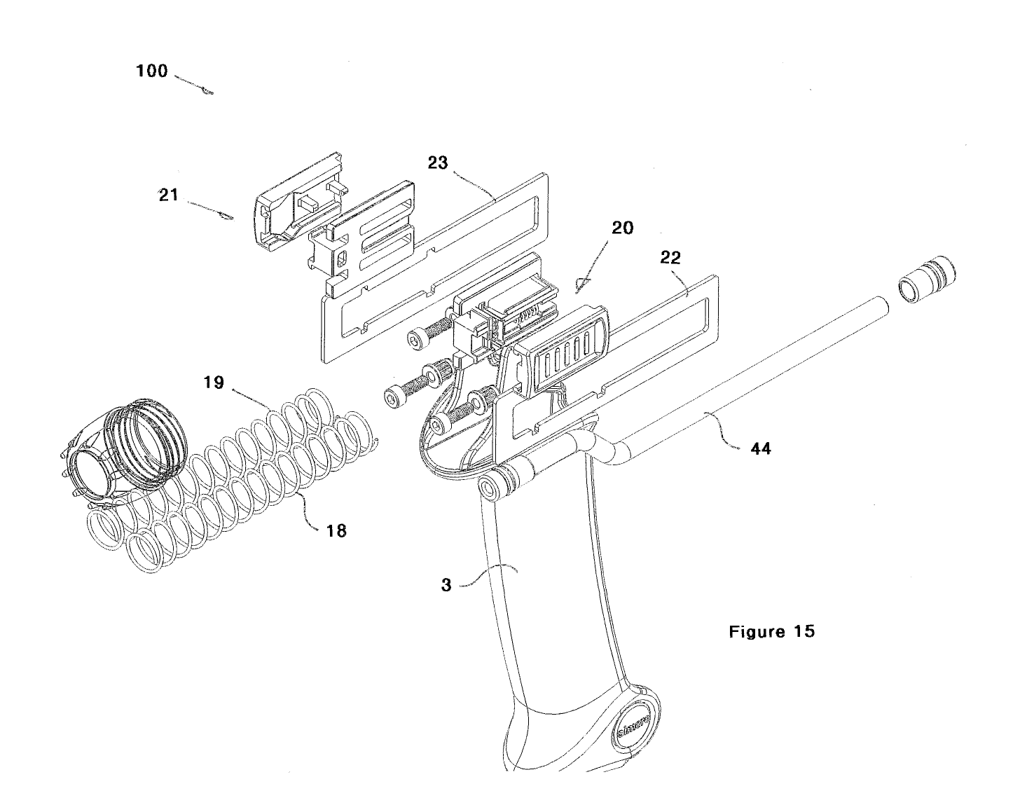

In another embodiment, shown in FIGS. 9 through 16 the inlet connections 45, 46 may be an integral part of a manifold provided adjacent the barrels at the first end 41. Reference numerals in these Figures, where they are the same, are for the same features.

Again, the applicator 100 comprises a housing 1 which is connected to or integral with a first handle 2, and has a first end 41 and second end 42. A second handle 3. Shown in the first position here, is pivotally connected at a lower end to the first handle 2. A manifold 54 contains the connections 45, 46 to the fluid supply (not shown), and the inlet and outlet valves (not shown) for the barrel 4, 5. In use, movement of the second handle 3 relative to the first handle 2, and thus the body or housing 1 activates the applicator 100, as is described above, to deliver fluid(s) from an outlet 40 at the second end 42. The second handle 3 drives a piston, one per barrel 4, to draw fluid into the barrel, and expel it out again. The dosage of the barrels can be adjusted using dosage control parts 20, 21. To prime the barrels there is a lever portion 38, and 39, to fill the barrel(s) with fluid. This is so there is minimal wastage when there are two or more barrels.

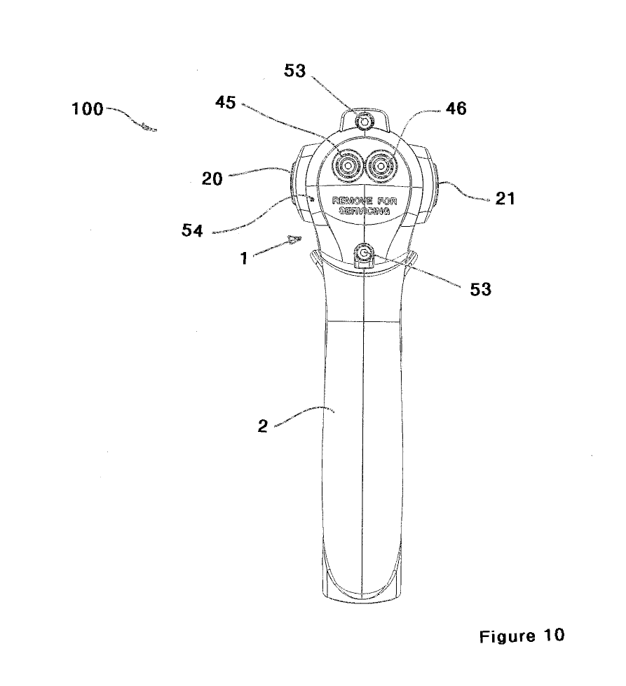

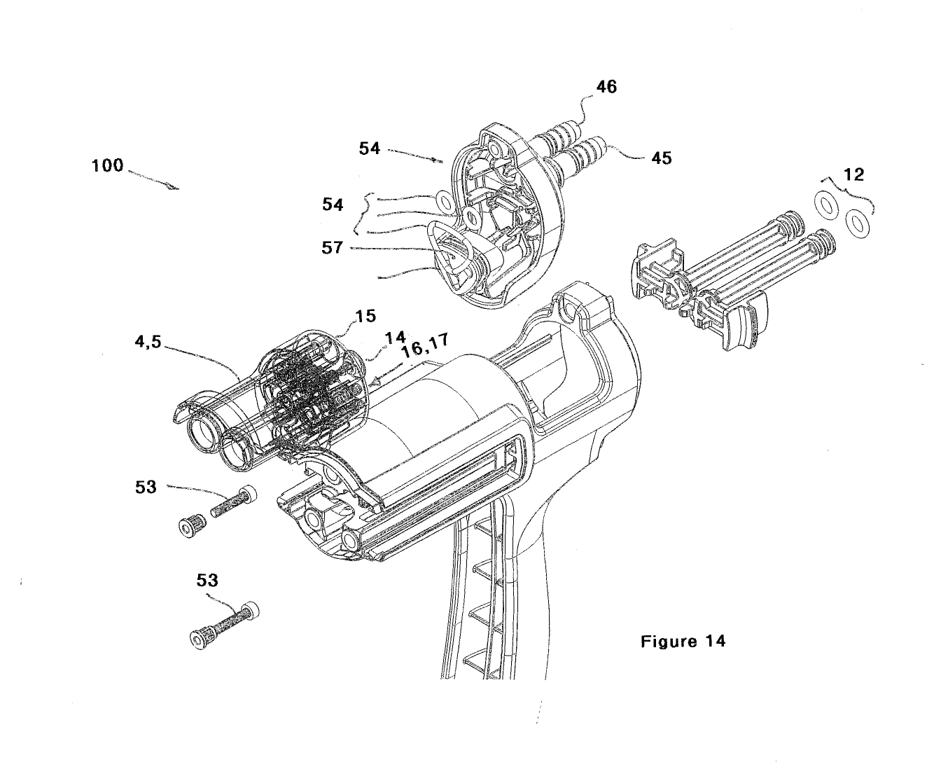

The manifold 54 is better shown in FIGS. 10 through 14. The manifold 54 can be attached in any re-connectable way, and in the preferred form uses a pair of fasteners 53 as shown in FIGS. 10, 13 and 14. Removing the fasteners allows the manifold to be removed from the first end 41 of the applicator 100, as shown in FIG. 14. Removing the manifold 54 allows access to the inlet valves 14, 15 (in this embodiment they are contained as part of the barrel 4,5 assembly), outlet valves 16, 17 and their respective seals 54. The mixing manifold 57 in this case is shown immediately downstream of the outlet valves 16, 17 and connects to the outlet conduit 44. Once the manifold 54 is removed, the barrel assembly 4, 5 can be extracted from the first end.

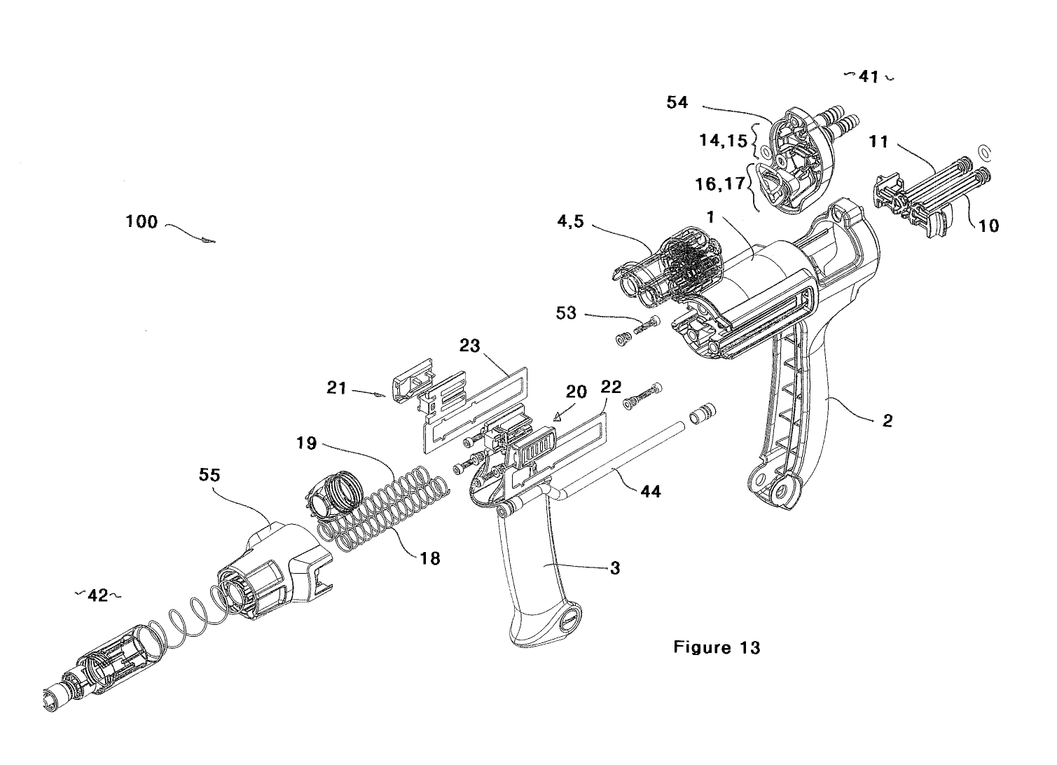

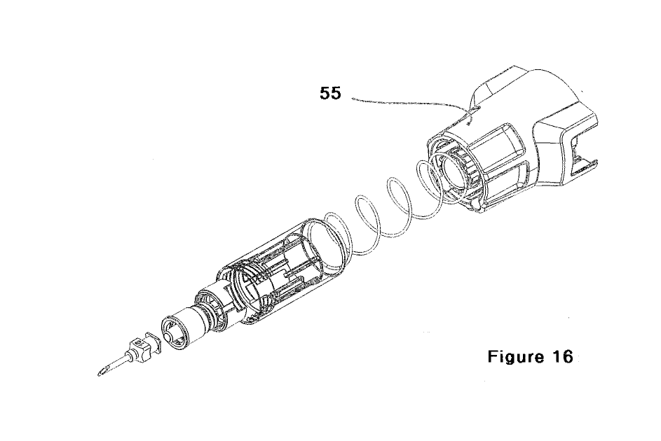

Likewise a housing front 55, shown in FIGS. 13 and 16 can be removed (again here with fasteners) from the body 1. This, in turn, allows for extraction of the piston rods 10, 11 and their pistons, seals, springs and abutment portions. Likewise the dosage control parts, and second handle 3 can be removed. In this way the entire applicator 100 can easily be assembled with minimal separate parts, to allow cleaning, removing of blockages and repair or replacement.

The applicator shown in the Figures and described above is configured as an injector.

However, it will be appreciated that other embodiments may be adapted for many other uses, including “pour-on” applicators, oral applicators, or nasal infusion applicators.

In some embodiments (not shown) the applicator may be configured as a “bottle mount” style injector, with a bottle mount fitting provided at the first end of the applicator. In this embodiment the (or each) inlet valve is provided adjacent the first end of the barrel. As is typical with bottle mount injectors, a short conduit portion may connect the inlet valve with an aperture in a side wall of the barrel.

While the invention has been particularly described with reference to a dual barrel applicator, in other embodiments the invention may be configured as a single barrel applicator, or as an applicator with more than two barrels (for example three barrels).

Throughout the claims, the words “comprise”, “comprising”, and the like, are to be construed in an inclusive sense as opposed to an exclusive or exhaustive sense, that is to say, in the sense of “including, but not limited to”.

Where in the foregoing description, reference has been made to specific components or integers of the invention having known equivalents, then such equivalents are herein incorporated as if individually set forth.

Although this invention has been described by way of example and with reference to possible embodiments thereof, it is to be understood that modifications or improvements may be made thereto without departing from the spirit or scope of the invention.

While the preferred embodiment of the invention has been illustrated and described, as noted above, many changes can be made without departing from the spirit and scope of the invention. Accordingly, the scope of the invention is not limited by the disclosure of the preferred embodiment. Instead, the invention should be determined entirely by reference to the claims that follow.

The embodiments of the invention in which an exclusive property or privilege is claimed are defined as follows:

1. An applicator comprising:
at least one barrel;
a piston moveable within the at least one barrel between a variable first position and a second position, wherein, in use, movement of the piston towards the first position can draw a first fluid into the barrel, and movement of the piston towards the second position can force the first fluid out of the barrel;
at least one fluid inlet valve to allow the fluid to flow into the at least one barrel at least under action of the piston, and at least one fluid outlet valve to allow the first fluid to flow out of the at least one barrel at least under action of the piston;
biasing means to bias the piston toward the first position;
a dosage control part that slides linearly on a longitudinal axis of the at least one barrel, and can lock in any one of a plurality of notches that each correspond to a dosage mark to abut an abutment portion which in turn defines the variable first position of the piston and therefore a dosage of the first fluid; and
piston actuating means operable to move the piston towards the second position to dispense the first fluid from the applicator.
wherein a user may set the dosage of the applicator via the dosage control part and deliver a dosage of the first fluid by the piston actuating means.

2. The applicator of claim 1 wherein the abutment portion is on, or connected to, the piston.

3. The applicator of claim 1 wherein the piston comprises a piston head and a piston rod connected to or integral with the piston head, wherein the piston rod comprises the abutment portion of the piston.

4. The applicator of claim 1 wherein the dosage control part is externally accessible and slidable by a user.

5. The applicator of claim 1 wherein the piston comprises a lever portion which is accessible from an exterior of the applicator.

6. The applicator of claim 5 wherein the lever portion allows a user to move the piston independently of the piston actuating means.

7. The applicator of claim 1 wherein the first fluid enters the at least one barrel at or adjacent a first end of the at least one barrel, and the first fluid exits the at least one barrel at or adjacent the first end.

8. The applicator of claim 7 wherein the at least one fluid inlet valve is adjacent one end of the at least one barrel, and the at least one fluid outlet valve is adjacent the same end of the at least one barrel.

9. The applicator of claim 8 wherein the at least one fluid inlet valve and the at least one fluid outlet valve are adjacent the first end.

10. The applicator of claim 9, further comprising an outlet, wherein the outlet is at a second end of the at least one barrel, opposite to the first end.

11. The applicator of claim 1, further comprising,
a second barrel;
a second piston moveable between a first position and a second position within the second barrel, wherein, in use, movement of the second piston towards the first position can draw a second fluid into the second barrel, and movement of the piston towards the second position can forces the second fluid out of the second barrel;
at least one second fluid inlet to allow the second fluid to flow into the second barrel; and
a second biasing means to bias the second piston toward the first position;
wherein the piston actuating means is operable to move both the first and second pistons towards the second position to dispense the first fluid and the second fluid from the applicator.

12. The applicator of claim 11, further comprising at least one second outlet valve to allow the second fluid to flow out of the second barrel.

13. The applicator of claim 11 wherein the at least one fluid outlet valve may be in fluid communication with the second barrel also.

14. The applicator of claim 11, further comprising a second dosage control part to abut a second abutment portion which in turn defines the first position of the second piston.

15. The applicator of claim 14 wherein the second piston comprises a second piston head and a second piston rod connected to or integral with the second piston head, wherein the second piston rod comprises the second abutment portion of the second piston.

16. The applicator of claim 11 wherein the second dosage control part is slideable along an axis which is substantially parallel to a longitudinal axis of the first and second barrels.

17. The applicator of claim 11 comprising a second lever portion which allows a user to move the second piston independently of the piston actuating means, and independently of the first piston.

18. The applicator of claim 11 wherein the first fluid and the second fluid may be the same or different, and may be delivered in the same or different dosages.

19. A method of using an applicator, comprising or including the steps of:
fluidly connecting supply of a first fluid to a manifold of the applicator;
adjusting a dosage of a first piston within a first barrel of the applicator using a first dosage control part, where the first dosage control part is slidingly lockable along a major axis of the first barrel in any one of a plurality of notches that each correspond to a dosage mark, the first dosage control part abutting a first abutment portion on the first piston which in turn varies a first position of the first piston and therefore the dosage of the first fluid;
priming the first barrel using a first lever portion;
forcing a first fluid from the first barrel by moving the first piston from the first position to a second position within the barrel, by use of a piston actuating means which, the first fluid moving from the first barrel via at least one fluid outlet valve; and
drawing the first fluid from the supply into the first barrel by the first piston moving from the second position back to the first position, the first fluid moving in via a first fluid inlet valve, the movement from the first position to the second position being biased;
wherein a user may set the dosage of the applicator via the first dosage control part and deliver a dosage of the first fluid by the piston actuating means.

20. The method of claim 19, further comprising the steps of:
fluidly connecting a supply of a second fluid to a manifold of the applicator;
adjusting a dosage of a second piston within a second barrel of the applicator using a second dosage control part, where the second dosage control part is slidingly lockable along a major axis of the second barrel, the second dosage control part abutting a second abutment portion on the second piston which in turn varies a first position of the second piston and therefore the dosage of the second fluid;
priming the second barrel using a second lever portion;
forcing a second fluid from the second barrel by moving the second piston from the first position to a second position within the barrel, by use of a piston actuating means which, the second fluid moving from the second barrel via at least one second fluid outlet valve; and
drawing the second fluid from the second supply into the second barrel by the second piston moving from the second position back to the first position, the second fluid moving in via a second first fluid inlet valve, the movement from the first position to the second position being biased;
wherein a user may set the dosage of the applicator via the second dosage control part and deliver a dosage of the second fluid by the piston actuating means.
